(19) United States
(12) Patent Application Publication
(10) Pub. No.: US 2024/0251729 A1
(43) Pub. Date: Aug. 1, 2024

(54) MAIZE POLLEN STORAGE AND CARRIER

(71) Applicant: Syngenta Crop Protection AG, Basel (CH)

(72) Inventors: Jay Austin Dinwiddie, RTP, NC (US); Sarah Hill-Skinner, RTP, NC (US); Jared Carter, RTP, NC (US)

(73) Assignee: Syngenta Crop Protection AG, Basel (CH)

(21) Appl. No.: 18/290,521

(22) PCT Filed: Jun. 15, 2022
(86) PCT No.: PCT/US22/33575

§ 371 Date: Nov. 14, 2023

Related U.S. Application Data
(60) Provisional application No. 63/289,299, filed on Dec. 14, 2021.
(60) Provisional application No. 63/214,384, filed on Jun. 24, 2021.

(51) Int. Cl. A01H 1/02

(52) U.S. Cl. CPC A01H 1/02

(57) ABSTRACT

Maize pollen is notoriously fragile and susceptible to degradation unless adequately stored. Unlike some tree pollen, which can be quite hardy and capable of successful fertilization for months or years after it is shed, maize pollen remains viable for mere hours after shedding before it begins to degrade. Described here is an invention for storing maize pollen where the pollen is collected and stored in a refrigerated, but not frozen, environment. Pollen stored as described herein may remain viable for twelve days, or two weeks, or longer. Adding a carrier compound can extend viability of the pollen.

### FIELD OF THE INVENTION

This invention relates to the field of maize breeding and human-induced pollination, and particularly the field of collecting, storing, and applying stored maize pollen in maize production fields and greenhouses.

### BACKGROUND

Pollen storage has long been both a need and a goal for plant breeders. See generally W. M. King, Report of chief on seed divisions, In REPORT OF THE COMMISSIONER OF AGRICULTURE (YEARBOOK), Washington D.C., GPO, 47-61 (1885) (articulating the desire for stored pollen “so that we might use it when and where convenient to ourselves.”). In some plants, pollen is quite hardy and long-lived. For example, gingko tree pollen can be collected and stored for six months or more with no specific care required. In contrast, other plants have pollen that is fragile and susceptible to rapid decay within hours if left exposed to the elements. Maize (corn) is one such plant.

In maize commercial hybrid production fields, current practice is to alternate four rows of female inbred plants with two rows of male inbred plants. The females are detasseled to prevent self-pollination, while the males are grown solely for their ability to pollinate the neighboring females. This arrangement works best where the female plants and the male plants are of similar maturity groups—that is, the males shed pollen at about the same time the females are receptive to the pollen.

However, a risk with current practices is a possibility of unsuccessful pollination, and therefore the loss of a crop, if the males and the females are of different maturity groups. Without pollen storage, the grower risks having the male plant shed pollen too early or too late and could lose an entire field due to failed pollinations. With pollen storage, pollen could be delivered at precisely the right time regardless of flowering time challenges. Interbreeding different maturity groups could be more easily accomplished, thus expanding the genetic pool and improving maize plant breeding, for example, by making maize lines that are more drought and/or disease resistant.

### SUMMARY

Growers need an ability to reliably collect and store maize pollen on one day or in one location and deliver that pollen to a field of females another day or at another location. To meet this need, a method of storing maize pollen is provided. In one embodiment, one collects an amount of fresh maize pollen; optionally treats the collected pollen with a carrier; seals the pollen in a container and optionally sets a vessel pressure to the container; and stores the pollen in a refrigerated environment. Pollen collected and stored in this manner remains viable for up to 20 days, and at least up to 12 days. In one aspect, the carrier is talc powder, or silica powder. In another aspect, the carrier is a metallic powder or mica. The carrier may be applied in apollen:carrier ratio of 1:2; 1:1, 2:1, 3:1, 4:1, 5:1, 6:1, 7:1, 8:1, 9:1, 10:1, 20:1, 30:1, 40:1, 50:1, and any ratio between 1:2 and 50:1. Preferably, the pollen:carrier ratio is 2:1. In another aspect, once placed in a sealable container, for example, a canning jar, the container is sealed and a vessel pressure is applied to the container until vessel pressure is between 1 atm and 0.01 atm, or between 0.6 atm and 0.3 atm, or between 0.4 atm and 0.35 atm. In another aspect, a carbon dioxide sequestering agent is added to the sealable container. In another aspect, the amount of pollen collected can be about 1 mg to about 54 g. In another aspect, the amount of pollen collected can be about 1 mL to about 150 mL or more. In another aspect, the amount of pollen collected is any desired amount.

### BRIEF DESCRIPTION OF THE FIGURES

FIG. 1 shows the seed set of four ears after pollination. The first ear (far left) was pollinated with fresh pollen. Pollen used to pollinate all other ears was stored in a similar manner: pollen was mixed in a 2:1 ratio with talc, and then 0.5 mL of pollen/talc mix was stored on aluminium pans in 125 mL glass vessels. Vessels were sealed, and then a vacuum was applied until the vessel pressure reached 0.4 atm before storage at 6° C. The second-to-left ear was pollinated with pollen stored for 3 days. The third-to-left ear was pollinated with pollen stored for 5 days. The fourth ear (far right) was pollinated with pollen stored for 7 days.

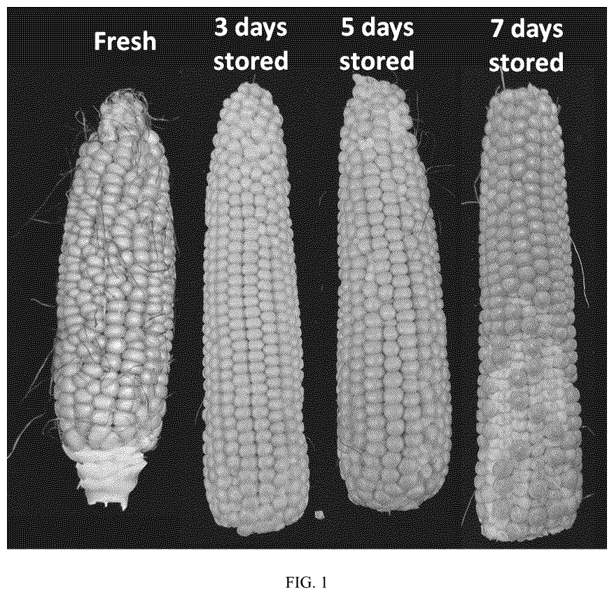

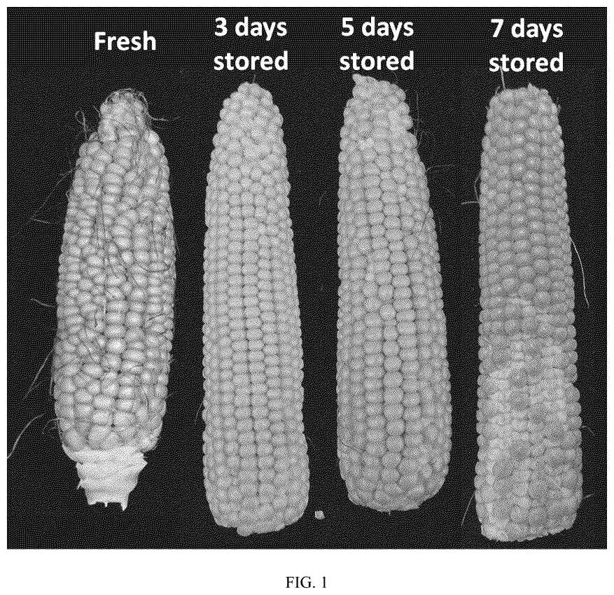

### DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

“Carbon dioxide sequestration,” as used herein means carbon dioxide (“CO₂”) is captured by way of a carbon dioxide sequestering agent, e.g., soda lime, activated carbon, ethanolamine, Zeolite 4A, lithium hydroxide (LiOH), or activated magnesium silicate (e.g., FLORISIL®). In this manner, excessive CO₂ buildup in a chamber is prevented. Optionally, a sequestration agent will prevent CO₂ from exceeding 10 mmol CO₂ per liter of chamber headspace.

“Carrier,” as used herein, means a compound, preferably in powdered form, which acts as an agent to accompany collected pollen. Suitable carrier compounds can be, but are not limited to, talc powder, silica powder, and the like.

“Clumping,” “Aggregating,” and similar terms, as used herein, refers to the tendency of pollen to bind together, whether due to excess moisture or other cause, in the absence of a carrier and/or suitable storage conditions. Pollen that has clumped is not flowable and cannot be blown by air onto a silk. Clumped pollen is unlikely to adhere to a silk sufficiently to cause pollination.

As used herein, the term “comprising” or “comprise” is open-ended. When used in connection with a method comprising a series of steps, that method is still practiced so long as the series of steps are performed, even if additional steps are performed.

“Crystalline silica,” as used herein, refers to a powdered form of silica derived from quartz or other natural rock formations. The terms “crystalline silica,” “SiO₂,” and “polycrystalline silica” are used interchangeably throughout. Crystalline silica has different structural properties than talc or amorphous silicas, which include but are not limited to a higher Mohs mineral hardness, higher bulk density, and lower specific surface area. In one embodiment, the crystalline silica comprises an average particle size between 1 nanometer (1 nm) and 100 micrometers (100 μm). In another embodiment, the crystalline silica comprises an average particle size between 1 micrometer (1 μm) and 10 micrometers (10 μm). Unless otherwise specified, particle size values provided herein are the average size.

“Activated magnesium silicate” as used herein, refers to a synthetic powdered magnesium silicate. The terms “activated magnesium silicate,” “synthetic amorphous activated magnesium silicate,” and “MgO₃Si” are used interchangeably throughout. “FLORISIL®” is a commercially available source of activated magnesium silicate. See www.ussilica.com/products/florisil. Activated magnesium silicate is characterized by an amorphous structure and high specific surface area. In one embodiment, the activated magnesium silicate comprises an average particle size between 75 micrometers (75 μm) and 149 micrometers (149 μm). In another embodiment, the activated magnesium silicate comprises an average particle size of less than 75 micrometers (<75 μm).

As used herein, the term transgenic “event” refers to a recombinant plant produced by transformation and regeneration of a single plant cell with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Thus, for example, “event 3272”, “3272” or “3272 event” as used herein, means the original 3272 transformant and/or progeny of the 3272 transformant and/or plants derived in any way from the original 3272 transformant. For 3272, See WO06/098952.

Other examples of transgenic events include, but are not limited to, MIR162 (See WO07142840), Bt11 (See U.S. Pat. No. 6,114,608 (construct) and WO8705629 (gene)), GA21 (See WO9704103 (gene) WO9844140 (cassette)), MIR604 (See WO05103301), MZIR098 (See WO18231890), 5307 (See WO10077816), DAS40278 (See U.S. Pat. No. 8,598,413), TC1507 (See WO04099447), DAS-59122-7 (See WO06/039376), NK603 (See U.S. Pat. No. 6,825,400), MON810 (See U.S. Pat. No. 6,713,259), MON863 (See U.S. Pat. No. 7,705,216), MON89034 (See WO07140256), MON88017 (See WO05059103), DP-4114 (See WO11084621), and MON87411 (See WO13169923).

“Flowable,” as used herein, means the ability of a powder-like substance to be easily moved by application of air, wind, or sound, or to be poured with unbroken continuity and proceed steadily and easily.

“Heterotic group,” as used herein, refers to a breeding categorization of inbred lines. “Heterotic group” and “heterotic pool” are used interchangeably and refer to the relationship between breeding pools of maize populations. Broadly, the primary designations for heterotic pool are: Stiff Stalk (“SS,” also called Iowa Stiff Stalk Synthetic, or “BSSS”), Non Stiff Stalk (“NSS”), and Iodent (“IDT”). See J. v. Hweerwaarden, et al., Historical genomics of North American maize, PROC. NAT'L ACAD. SCI. U.S.A. 109(31): 12420-25 (2012). These are not exclusive, however, and other designations are known, e.g., Lancaster Sure Crop (“LSC”). See, e.g., C. Livini, et al., Genetic diversity of maize inbred lines with and among heterotic groups revealed by RFLPs, THEOR. APPL. GENET. 84: 17-25 (1992). See further Hallauer et al. (1998) CORN BREEDING, p. 463-564; G. F. Sprague and J. W. Dudley (ed.) CORN AND CORN IMPROVEMENT; Smith, et al. (1990) Theor. Appl. Gen. 80:833-840; Mikel and Dudley (2006) Crop Set 46: 1193-1205. See also WO2020/205334 and WO2021/041077, incorporated herein by reference in their entireties.

The term “germplasm” refers to the totality of the genotypes of a population or other group of individuals (e.g., a species or plant line). The phrase “adapted germplasm” refers to plant materials of proven genetic superiority; e.g., for a given environment or geographical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, the term “mica” refers to a group of minerals generally having the chemical formula X₂Y₄₋₆Z₈O₂₀(OH, F)₄, in which X is an alkali metal or alkaline earth metal, Y is a transition metal, post-transition metal, or alkaline earth metal, and Z is silicon, aluminum, or may include other transition metals.

“Starting oxygen content,” as used herein, refers to the amount of oxygen present (whether measured as an absolute measurement, a percentage, or otherwise) in the atmosphere of a chamber comprising collected pollen at its outset and once initially sealed. In one embodiment, the starting oxygen content is between 0.12 mmol O₂/g pollen/day stored and 0.57 mmol O₂/g pollen/day stored, inclusive. In another embodiment, the starting oxygen content is between 0.24 mmol O₂/g pollen/day stored to 0.57 mmol O₂/g pollen/day stored, inclusive. “Starting oxygen content,” “Start mmol 02,” “Start mmol O₂/g pollen,” and “Start mmol O₂/g pollen/day stored” are used interchangeably herein.

A “plant” is any plant at any stage of development, particularly a seed plant. In particular, in the context of this disclosure, a plant refers to a maize plant. As used herein, the term “plant line” refers to a single plant material or a genetically identical set of materials

“Platform,” as used herein, means a surface within a container which is in direct contact with the pollen and carrier mixture, and which prevents direct contact with the container itself. For example, the platform may be filter paper or an aluminum tray.

“Pollen:Carrier Ratio,” as used herein, means the proportion of pollen present in a mixture with a carrier. For example, and not by way of limitation, a mixture of pollen and carrier with a pollen:carrier ratio of 2:1 comprises 2 parts pollen measured by weight or volume and one part carrier compound, e.g., talc, measured by weight or volume.

“Refrigerated environment,” as used herein, means any condition where the temperature is less than ambient temperature (or room temperature), but does not fall below the temperature at which water freezes. Said another way, if ambient temperature is 25° C., then a refrigerated environment comprises temperatures greater than 0° C. and less than 25° C. Likewise, a refrigerated environment comprises temperatures between 2° C. and 10° C.

“Sealable container,” as used herein, means any container capable of forming an air-tight seal. Preferably, a sealable container is also capable of holding a vacuum.

“Seed Set,” as used herein, means the number of kernels produced on a cob from a successful pollination. Seed set may be expressed qualitatively (e.g., low, good, or high) or quantitatively. In a quantitative measurement, the measurement may be given as either a percentage or a number of seeds per ear. The term generally refers to the percentage or number of normal kernels (i.e. non-aborted, endosperm-viable kernels). For normal maize lines (i.e. not haploid inducer lines), a seed set above 80% (or above 300 kernels per ear) is considered a good seed set. Achieving a good seed set is a goal of a controlled pollination.

“Storage,” as used herein, refers to the act of storing pollen for a suitable period. A suitable storage period may be as little as 24 hours or as much as 12 days.

“Treatment,” as used herein, means intentional application of compounds or environmental constraints to pollen. In particular, a pollen treatment may include addition of a carrier compound to the pollen to preserve the pollen's flowability and viability.

“Vessel pressure,” as used herein, refers to any artificially imposed atmospheric pressure within the vessel. Vessel pressure values are measured here in units of standard atmosphere “atm,” e.g., 0.5 atm, however, other units may be used (e.g., Torr or Pascal or “Pa;” 1 Pa=9.8692×10⁻⁶ atm) as desired to measure vessel pressure. It is expressly contemplated that vessel pressure may meet or exceed 1 atm. Under conditions where the vessel pressure is between 0 and 1 atm, “vessel pressure” and “vacuum” possess the same meaning and are used interchangeably. “Vessel pressure” is the preferred term, however, as it contemplates both vacuum conditions and conditions where the artificially imposed atmospheric pressure exceeds ambient atmosphere (e.g., 1 atm). In some examples, the vessel pressure may be 1 atm or 2 atm or 3 atm absolute. In some examples, the vessel may be pressurized with pure oxygen gas at 18 mmol, 24 mmol, or 27 mmol O₂ per liter of storage vessel headspace.

“Vigor,” as used herein, means the ability of pollen to adhere to silks, germinate pollen tubes, and successfully fertilize egg cells. “Viable,” “Viability,” and similar terms, are used interchangeably with “Vigor.”

### DETAILED DESCRIPTION

Producibility in maize seed production (i.e., a measure of whether the required quantities of inbred or hybrid seed can be produced through self-pollination or cross pollination at an economical cost that does not exceed the value of the seed being produced) is a critical factor for success in developing maize inbred parent lines, as large quantities of inbred parent line seed are required to produce the hybrid seed sold to customers. A maize inbred parent line with low producibility may be discontinued due to excessive costs in parent seed production, even if that inbred parent line can produce hybrids with characteristics that are desirable to customers (e.g., leading GM and genome edited traits, high yield, disease resistance). Pollen storage technology can be used to enhance the producibility of inbred maize parent lines used in hybrid seed production.

Challenges to producibility that may be addressed by pollen storage technology include but are not limited to, low pollen production, low total pollen shed, short duration of pollen shed, short duration of silk receptivity, and GM or genome edited traits that may impact plant reproductive characteristics. An additional challenge with self-pollination may be a long self-split, which is defined by the number of days between when pollen starts shedding and when silks emerge and become available for pollination. In some iterations, self-split can be a negative value, where silks emerge for pollination before the start of pollen shed. The observed self-split may be a result of the inbred parent line genetics or a result of stress in the growing environment that reduces the rate of silk extension and increases the number of days between start of pollen shed and silk availability for pollination.

To address these producibility challenges, pollen storage technology may be used to collect pollen during the optimal window for pollen shed, store that pollen while maintaining pollen viability, then apply the pollen during the optimal window for silk emergence and receptivity. In some iterations, pollen collection may be conducted multiple times per day. In other iterations, pollen may be collected on multiple days throughout the duration of pollen shed. Application of stored pollen may use combined pollen collected over multiple days, and multiple applications may take place on the same day or across multiple days. Pollen application may use combined pollen collected from multiple field locations into a single application to one location. In some iterations, pollen may be collected in one geography and applied to silks in a different geography. The geographies may be different fields at the same production location, fields in different states or municipalities within country, or fields in different countries. In some iterations, pollen is collected from temperate maize inbred parent lines grown in a temperate location and applied to sub-tropical or tropical maize inbred parent lines grown in sub-tropical or tropical locations. In other iterations, pollen is collected from sub-tropical or tropical maize inbred parent lines grown in sub-tropical or tropical locations and applied to temperate maize inbred parent lines grown in a temperate location. By addressing these challenges to producibility, pollen storage technology may enable seed increase for desirable maize inbred parent lines that will produce new hybrids with desirable characteristics for sale to customers. Pollen storage technology may also enable economical hybrid seed production for combinations of temperate, sub-tropical, and tropical maize inbred parent lines that are not currently feasible.

Accordingly, an embodiment provides a composition comprising maize pollen and crystalline silica. In one aspect of the composition, the crystalline silica comprises an average particle size. In another aspect, the average particle size is between about 1 nanometer and about 100 micrometers. In another aspect, the average particle size is between about 1 micrometer and about 10 micrometers. In yet another aspect, the maize pollen is 0 days old, 1 day old, 2 days old. 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, or more.

Another embodiment provides a method of storing viable maize pollen, comprising: a) collecting an amount of fresh maize pollen; b) optionally applying a carrier to the collected maize pollen of step a) to obtain an amount of treated maize pollen; c) placing the amount of fresh maize pollen or the amount of treated maize pollen in a sealable container and optionally setting a vessel pressure; and d) storing the product of step c) in a refrigerated environment. In one aspect of the method the stored maize pollen remains viable for at least 1, 2, 3, 4, 5, 6, 7, 8, 9, 10, 11, 12, 13, 14, 15, 16, 17, 18, 19, or 20 days. In one aspect, the container comprises a volume of 1 mL to 100 L, or the container comprises a volume of 10 mL to 20 L, or the container comprises a volume of approximately 12 L, approximately 1.8 L, approximately 1 L, 500 mL, or approximately 125 mL. In another aspect of the method, the amount of fresh maize pollen or treated maize pollen is at least approximately 54 g, or at least approximately 25 g, or at least approximately 11 g, or at least approximately 720 mg, or at least approximately 360 mg, or at least approximately 180 mg, or at least approximately 90 mg, or at least approximately 45 mg, or at least approximately 1 mg. In another aspect of the method, the vessel pressure is between approximately 0.6 atm and 0.3 atm, or the vessel pressure is between approximately 0.4 atm and 0.35 atm. In another aspect of the method, the carrier is selected from the group consisting of crystalline silica, activated magnesium silicate, talc, metallic powder, and mica mineral. In one aspect, the metallic powder is a metallic oxide powder or a metallic carbide powder. In another aspect, the metallic powder is of an average particle size. In one aspect, the average particle size is 10 μm spherical. In another aspect, the metallic powder is stainless steel powder.

In another aspect, the carrier is present in a pollen:carrier ratio selected from the group consisting of 1:20, 1:30, 1:10, 1:5, 1:3, 1:2, 1:1, 2:1, 3:1, 4:1, 5:1, 6:1, 7:1, 8:1, 9:1, 10:1, 20:1, 30:1, 40:1, 50:1, and any ratio between 1:20 and 50:1. In one aspect, the pollen:carrier ratio is 2:1. In another embodiment of the method, the sealable container comprises a platform. In one aspect, the platform comprises a material which reduces pollen clumping due to condensation formation. In another aspect, the platform is an aluminum tray, copper tray, nickel tray, or stainless-steel tray. In another embodiment, the sealable container comprises a material which reduces pollen clumping due to condensation formation. In another aspect, the sealable container is fabricated from glass, aluminum, acrylic, or stainless steel. In another embodiment of the method, the refrigerated environment comprises a temperature range selected from the group consisting of 1° C.-10° C., 4° C.-8° C. and 5.5° C.-6.5° C. In one aspect, the refrigerated environment comprises a temperature of approximately 6° C. In another embodiment of the method, the pollen is stored in the refrigerated environment for 20 or fewer days, 19 or fewer days, 18 or fewer days, 17 or fewer days, 16 or fewer days, 15 or fewer days, 14 or fewer days, 13 or fewer days, 12 or fewer days, 11 or fewer days, 10 or fewer days, 9 or fewer days, 8 or fewer days, 7 or fewer days, 6 or fewer days, 5 or fewer days, 4 or fewer days, 3 or fewer days, 2 or fewer days, or 1 day, or less than 1 day. In one aspect, the pollen is stored for 12 or fewer days. In another embodiment of the method, the sealable container comprises a starting oxygen content. In one aspect, the starting oxygen content is between 0.12 mmol O₂/g pollen/day stored and 0.57 mmol O₂/g pollen/day stored. In another aspect, the starting oxygen content is approximately 0.24 mmol 02/g pollen/day stored to 0.57 mmol O₂/g pollen/day stored. In another embodiment of the method, the sealable container comprises a CO₂ sequestration means. In one aspect, the sequestration agent is selected from the group consisting of activated charcoal, ethanolamine, Zeolite 4A, lithium hydroxide (LiOH), soda lime, calcium silicate (Ca₂O₄Si), and activated magnesium silicate (e.g., FLORISIL®).

Another embodiment provides a method of applying stored maize pollen to a stigma, comprising: a) obtaining stored maize pollen by the method above; b) applying the stored pollen to a silk; wherein the stored maize pollen is applied to the silk after collection. In another embodiment, the stored maize pollen is applied to the stigma at least 1 day after collection. In one aspect, the stigma is a maize silk. In another aspect, the maize silk is a different heterotic group than the heterotic group corresponding to the stored maize pollen. In yet another aspect, the maize silk is from a tropical or sub-tropical heterotic group and the stored maize pollen is from a temperate heterotic group; or the maize silk is from a temperate heterotic group and the stored maize pollen is from a tropical or sub-tropical heterotic group. In another aspect, the heterotic group is selected from the group consisting of Stiff Stalk, Non-Stiff Stalk, Iodent, and Lancaster. In another aspect, the maize silk is a different maturity group than the maturity group corresponding to the stored maize pollen. In yet another aspect, the stigma is a wheat stigma.

In another embodiment, the maize pollen being stored is transgenic maize pollen. In another aspect, the transgenic maize pollen comprises a transgenic event selected from the group consisting of MIR162, Bt11, GA21, MIR604, MZIR098, 5307, 3272, DAS40278, TC1507, DAS-59122-7, NK603, MON810, MON863, MON89034, MON88017, DP-4114, and MON87411. In one aspect, the transgenic maize pollen comprises transgenic events Bt11, GA21, and MIR162. In another aspect, the transgenic maize pollen comprises transgenic events Bt11 and MIR162. In yet another aspect, the transgenic maize pollen comprises transgenic event MIR162.

In another aspect, the vessel pressure used during the storage method is pressurized with standard atmospheric oxygen and pressurized with pure oxygen gas. In one aspect, the standard atmospheric oxygen is 1 atm absolute. In one aspect, the standard atmospheric oxygen is 2 atm absolute. In another aspect, the standard atmospheric oxygen 3 atm absolute. In another aspect, the pure oxygen gas is 18 mmol O₂ per liter of storage vessel headspace. In one aspect, the pure oxygen gas is 24 mmol O₂ per liter of storage vessel headspace. In another aspect, the pure oxygen gas is 27 mmol O₂ per liter of storage vessel headspace.

### Examples

### 1. Collection

Maize plants were grown in field and in greenhouse conditions. Once tassels emerged and had begun shedding pollen, bags were placed over the tassels to collect the pollen. Bags were typically placed during the late afternoon and removed the following morning. Collected pollen, after sifting away any anthers or other tassel material and optionally mixed with a carrier, was then placed in an appropriate, sealed container.

Alternatively, pollen is collected by harvesting the pre-shed tassels from the maize plants. The tassels can be placed in a beaker of water and allowed to shed pollen normally, or the tassels can be dried, macerated, and filtered to collect the pollen mechanically. See, e.g., U.S. Pat. No. 8,252,988 (filed Jun. 27, 2007), incorporated by reference herein in its entirety.

### 2. Microbial Growth

Microbial analyses were performed to understand whether bacteria or fungi are present in collected pollen in sufficient quantities to contribute to degradation of fresh pollen. The effects of microbial pressure were first observed visually in pollen stored at 23° C. All pollen samples were mixed in a 2:1 ratio of pollen:talc prior to storage. After 4 days of sealed storage at 23° C., stored pollen was covered in swathes of microbial colonies. To better understand the makeup of these microbes and how they varied with environmental conditions, 3M Petrifilm was used on fresh pollen from the greenhouse and the field. Specifically, the presence of molds, aerobic bacteria, lactic acid bacteria, and heterotrophic bacteria was assayed. Perhaps unsurprisingly, pollen collected from field-grown plants played host to more microbial pressure by several orders of magnitude (Table 1). Additionally, heterotrophic bacteria that were not present in greenhouse pollen were found in similar numbers to aerobic bacteria in field pollen. In both cases, the presence of molds was significant while the extent of presence of lactic acid bacteria remains unclear (Table 1).

TABLE 1
Colony forming units (CFU) of various microbes
in 50 uL of fresh greenhouse and field pollen.

| | Greenhouse | Field |
|---|---|---|
| | pollen | pollen |
| Microbe Type | (CFU) | (CFU) |
| Molds | 500 | 1,400 |
| Aerobic Bacteria | 32-80 | 20,200 |
| Lactic Acid Bacteria | 0-1000 | 0-1000 |
| Heterotrophic Bacteria | 0 | 19,000 |

The presence of these microbes could be controlled by anti-microbial treatment of the pollen or changes to the storage environment. As such, temperature was assayed as a means of effective control of microbial growth on stored pollen. 0.36 g of pollen was stored in a 2:1 mixture of pollen:talc for seven days at 23° C., 6° C., and 2° C. in 125 mL sealed glass vessels at 1 atm pressure, and O₂ consumption and CO₂ production within the sealed storage vessels were recorded each day. At 4 days of storage, the 23° C. samples no longer germinated pollen tubes on media nor stained with the enzymatic stain, MTT (Tables 2 and 3). As noted previously, visible microbial colonies had also grown on these samples. Conversely, the 6° C. and 2° C. samples did not show any visible signs of microbial growth and successfully germinated pollen tubes and stained with MTT out to 6 days of storage (Tables 2 and 3). At seven days of storage, the 2° C. samples no longer germinated, though stained positively for MTT—showing how the ability to germinate tubes fails prior to the complete loss of enzymatic activity in dying pollen.

TABLE 2
Observation of pollen tube germination in pollen
samples stored at different temperatures.

| Days | Temperature | | |
|---|---|---|---|
| stored | 23° C. | 6° C. | 2° C. |
| 1 | Yes | Yes | Yes |
| 2 | Yes | Yes | Yes |
| 3 | Yes | Yes | Yes |
| 4 | No | Yes | Yes |
| 5 | No | Yes | Yes |
| 6 | No | Yes | Yes |
| 7 | ND | Yes | No |

ND = no data

TABLE 3
Observation of enzymatic staining in pollen
samples stored at different temperatures.

| Days | Temperature | | |
|---|---|---|---|
| stored | 23° C. | 6° C. | 2° C. |
| 1 | Yes | Yes | Yes |
| 2 | Yes | Yes | Yes |
| 3 | Yes | Yes | Yes |
| 4 | No | Yes | Yes |
| 5 | No | Yes | Yes |
| 6 | No | Yes | Yes |
| 7 | ND | Yes | Yes |

ND = no data

Over the course of the first 6 days of storage, oxygen consumption and CO₂ production in samples stored at 6° C. and 2° C. was less than 17% of that observed for samples stored at 23° C. (Table 4). This difference is attributed to forced reduction in the metabolic rate of both the pollen and microbes at lower temperatures. This is underlined by the lack of visible microbial growth in the 6° C. and 2° C. samples that is seen in the 23° C. samples after 4 days of storage. Further reduction in metabolic rate was observed between samples stored at 6° C. compared to samples stored at 2° C. while general vigor was similar up to 6 days (Tables 4, 2, and 3). These results together show that reduced temperatures impede microbial respiration and act as an effective treatment against undesirable microbial growth on stored pollen.

TABLE 4
Final amounts of O₂ and CO₂ at 6 days of storage and rate
of O₂ usage and CO₂ production at various temperatures.

| Temp | Mean mmol | Mean mmol | mmol O₂ | mmol CO₂ |
|---|---|---|---|---|
| (° C.) | O₂ Used | CO₂ Made | Used/Day | Made/Day |
| 2 | 0.18 | 0.16 | 0.03 | 0.02 |
| 6 | 0.28 | 0.25 | 0.04 | 0.04 |
| 23 | 1.07 | 1.03 | 0.15 | 0.15 |

### 3. Storage Temperature

As shown above, storage temperature clearly plays an important role in determining pollen and microbial metabolic rate during storage. To test temperature's effects on pollen vigor, pollinations were also performed. Reductions in seed set occurred across time for all temperatures tested. However, the reduction in seed set over time was amplified at 2° C. and 23° C. relative to 6° C. (Table 5). To further assess the impact of temperature on pollen vigor, an additional experiment was conducted with storage temperatures ranging from approximately 4.3-8.7° C. (Table 6) for 7 days. For all treatments, 0.18 g pollen was mixed with talc in a 2:1 pollen:talc ratio prior to storage in 125 mL glass vessels at 0.4 atm. Combined with the data from Table 5, reductions in seed set occurred at temperatures below 4° C. and above 6° C., with the most dramatic reductions observed at 2° C. and 23° C. No temperature tested for pollinations was completely prohibitive to successful storage but instead impacted the longevity of storage (Tables 5 and 6). In conclusion, these studies show that temperatures from at least 2° C. to 23° C. are permissible for pollen storage, though longer storage durations may be achieved at lower temperatures around 2-8° C.

TABLE 5
Seed set decay over time across different storage temperatures.

| Storage | Days | Avg Seed | |
|---|---|---|---|
| Temp (° C.) | Stored | Set/Ear (k) | Stdev (k) |
| 2 | 2 | 257 | 173 |
| 2 | 3 | 150 | 158 |
| 2 | 4 | 33 | 8 |
| 2 | 5 | 65 | 37 |
| 2 | 6 | 15 | 3 |
| 6 | 2 | 367 | 44 |
| 6 | 3 | 83 | 2 |
| 6 | 4 | 153 | 16 |
| 6 | 5 | 99 | 33 |
| 6 | 6 | 40 | 13 |
| 23 | 2 | 27 | 8 |
| 23 | 3 | 3 | 3 |

TABLE 6
Seed set from pollen stored for 7 days
at various storage temperatures.

| | Storage | Avg Seed | |
|---|---|---|---|
| Days Stored | Temp (° C.) | Set/Ear (k) | Stdev (k) |
| 7 | 4.3 | 77 | 53 |
| 7 | 6 | 89 | 44 |
| 7 | 8.7 | 34 | 30 |

### 4. Oxygen and Carbon Dioxide

As shown in example 2, pollen is still metabolically active during storage—utilizing O₂ and producing CO₂ during aerobic respiration. As the pollen continues to respire during storage, O₂ w ill necessarily be depleted while CO₂ will build up. To ascertain the impacts these changes in vessel atmosphere may have on the pollen, several experiments were conducted. In the first, several weights of pollen were stored as 2:1 pollen:talc mixes in standard 125 mL vessels (e.g., 4 oz Ball mason jars) at 0.4 atm at 6° C. with starting oxygen of 0.44 mmol (20.9%) and starting CO₂ of 0.00084 mmol (0.04%). After 7 days of storage, endpoint oxygen and CO₂ readings were measured (Table 7). Samples at or above 0.54 g depleted oxygen below 0.1 mmol and showed no tube germination on media. Samples exceeding 0.54 g had nearly depleted all available oxygen and failed to stain using MTT—indicating complete lack of viability. In this same experiment, CO₂ was observed to accumulate in a negative linear pattern to oxygen depletion. Thus, by the end of the 7-day storage period, samples which had mostly exhausted available oxygen had accumulated over 0.4 mmol of CO₂.

TABLE 7
Oxygen usage and CO₂ production from various
weights of pollen stored for 7 days.

| Pollen | Mean End | Stdev End | Mean End | Stdev End | MTT | Germin- |
|---|---|---|---|---|---|---|
| Wt (g) | mmol O₂ | mmol O₂ | mmol CO₂ | mmol CO₂ | Stain | ation |
| 0.18 | 0.33 | 0.02 | 0.14 | 0.01 | Yes | Yes |
| 0.36 | 0.20 | 0.03 | 0.27 | 0.02 | Yes | Yes |
| 0.54 | 0.10 | 0.03 | 0.39 | 0.03 | Yes | No |
| 0.72 | 0.04 | 0.01 | 0.48 | 0.01 | No | No |
| 0.90 | 0.04 | 0.00 | 0.50 | 0.01 | No | No |
| 1.08 | 0.04 | NA | 0.51 | NA | No | No |

To test the effect of CO₂ accumulation on pollen vigor, a second experiment was conducted. In this experiment, 125 mL vessels containing 0.36 g of pollen mixed with 0.18 g of talc were spiked with increasing amounts of CO₂ prior to storage (while maintaining total vessel pressure at 1 atm) and then stored at 6° C. Then, once a day over a 6 day period pollen tube germination for each treatment was assayed on a scale of 1-5 (Tables 8 and 9). As expected, the baseline starting CO₂ (approximately 0.002 mmol, the amount present in 125 mL of natural atmosphere at room temperature) resulted in good germination throughout the 6 day storage period. Pollen longevity negatively responded to increased starting CO₂, such that all vessels with greater than atmospheric starting CO₂ showed lowered or loss of tube germination earlier than the baseline (Table 9). These results demonstrate the toxicity of accumulated CO₂ on pollen grains and the importance of its control and/or sequestration during storage.

TABLE 8
Germination rating scale.

| Scale | Gemination Rate |
|---|---|
| 5 | >60% |
| 4 | 41-60% |
| 3 | 21-40% |
| 2 | 1-20% |
| 1 | 0% |

TABLE 9
Pollen tube germination over time for pollen stored
with increasing amounts of starting CO₂.

| Approx. | Germination Rate at Days Stored | | | | | | Approx. | |
|---|---|---|---|---|---|---|---|---|
| Starting mmol | One | Two | Three | Four | Five | Six | Starting mmol | End mmol |
| CO₂ | Day | Days | Days | Days | Days | Days | O₂ | O₂ |
| 0.002 | 5 | 5 | 5 | 5 | 4 | 4 | 1.08 | 0.74 |
| 0.41 | 5 | 5 | 4 | 3 | 2 | 1 | 0.99 | 0.70 |
| 1.03 | 5 | 4 | 2 | 1 | 1 | 1 | 0.80 | 0.57 |
| 3.10 | 4 | 1 | 1 | 1 | 1 | 1 | 0.43 | 0.40 |
| 4.65 | 1 | 1 | 1 | 1 | 1 | 1 | 0.09 | 0.13 |

To further explore metabolic rate within the storage vessels, we calculated the average O₂ usage and CO₂ production from 245 individual samples. All samples were stored for 5-7 days at 6° C. with a 2:1 pollen:talc ratio. Samples were included only if their germination rating was 2 or above at the end of storage. Samples were stored under a range of pressures ranging from 0.13-1 atm and with a range of pollen amounts 0.09-25 g. For pollen undergoing aerobic respiration, O₂ and CO₂ are exchanged in a 1:1 stoichiometry. As a result, the amount of oxygen used and the amount of CO₂ produced during aerobic respiration is approximately equivalent. Based on the 245 samples, we found average O₂ usage and CO₂ production/g pollen/day to be around 0.17 mmol (Table 10). Maximum rate was nearly double at 0.30 mmol/g pollen/day and minimum was as low as 0.01 mmol/g pollen/day.

TABLE 10
Average, maximum, and minimum metabolic
rates of pollen during storage.

| | mmol O₂ Used/mmol |
|---|---|
| | CO₂ Produced/g |
| | Pollen/Day |
| Average | 0.17 |
| Stdev | 0.04 |
| Max | 0.30 |
| Min | 0.01 |

After showing CO₂ accumulation is toxic to pollen during storage, our next step was to identify a way to safely sequester CO₂ passively during storage. There are numerous chemical compounds that actively bind CO₂ and remove it from the atmosphere. In a new experiment, we tested the ability of soda lime to sequester CO₂ in the storage vessel setup. Soda lime binds CO₂ in a reaction that utilizes water vapor and produces CaCO₃ and heat. As such, water vapor within the storage environment is expected to be consumed as CO₂ is produced by pollen and subsequently sequestered by the soda lime. To offset this loss of water vapor, we also tested adding liquid water to the storage vessels. In each 125 mL vessel, starting oxygen was approximately 1 mmol, starting CO₂ was approximately 0.002 mmol, and vessel pressure was 1 atm. Vessels were tested with/without 0.05 g of soda lime and with/without 10 mL of water. Pollen amount was also varied from 0.18 g to 0.72 g, but all pollen was mixed with talc in a 2:1 pollen:talc ratio prior to 5 days of storage at 6° C. Pollen moisture content (PMC) was measured as the difference between fresh weight and dry weight. Initial pollen moisture content for this experiment was 53%.

For all pollen amounts, vessels that contained soda lime showed positive sequestration of CO₂ as reflected in the lower remaining mmol of CO₂ relative to the same weight of pollen in vessels without soda lime (Table 11). Due to detection limits of the instrument used, it was not possible to detect a complete absence of CO₂, so samples with 0.01 mmol CO₂ remaining were considered to have all CO₂ sequestered. For pollen amounts greater than 0.36 g, 0.05 g of soda lime was insufficient to completely sequester all CO₂ over the 5 day period. As such, the amount of soda lime must be scaled to the amount of pollen in the container (about 0.02 g soda lime/g pollen/day). In vessels not containing supplemental water, PMC is significantly reduced—the likely result of moisture being drawn from the pollen into the atmosphere to restabilize humidity as water vapor is utilized by soda lime (Table 11). Interestingly, vessels containing water showed a slight increase in final PMC relative to initial PMC. This may indicate a slow uptake of water vapor by the pollen over time. The results of this experiment together show the effectiveness of soda lime as a CO₂ sequestering agent for pollen storage and the importance of a secondary source of water vapor to offset the drying effects of the soda lime reaction.

TABLE 11
CO₂ sequestration in vessels with/without soda lime and water
after 5 days of storage with various amounts of pollen.

| Pollen Wt | Added Soda | Added | Remaining | Remaining | Final | MTT | |
|---|---|---|---|---|---|---|---|
| (g) | Lime | H₂O | mmol O₂ | mmol CO₂ | PMC | Stain | Germination |
| 0.18 | Yes | Yes | 0.80 | 0.01 | 54 | Yes | Yes |
| 0.36 | Yes | Yes | 0.60 | 0.02 | 56 | Yes | Yes |
| 0.54 | Yes | Yes | 0.44 | 0.06 | 54 | Yes | Yes |
| 0.72 | Yes | Yes | 0.33 | 0.24 | 54 | Yes | Yes |
| 0.18 | Yes | No | 0.92 | 0.01 | 38 | Yes | Yes |
| 0.36 | Yes | No | 0.76 | 0.01 | 46 | Yes | Yes |
| 0.54 | Yes | No | 0.51 | 0.20 | 50 | Yes | Yes |
| 0.72 | Yes | No | 0.46 | 0.14 | 50 | Yes | Yes |
| 0.18 | No | Yes | 0.75 | 0.18 | 56 | Yes | Yes |
| 0.36 | No | Yes | 0.58 | 0.33 | 56 | Yes | Yes |
| 0.54 | No | Yes | 0.49 | 0.40 | 55 | Yes | Yes |
| 0.18 | No | No | 0.87 | 0.17 | 43 | Yes | Yes |
| 0.36 | No | No | 0.72 | 0.31 | 46 | Yes | Yes |
| 0.54 | No | No | 0.61 | 0.41 | 50 | Yes | Yes |

After confirming the effectiveness of soda lime in sequestering released CO₂ in the storage vessel, we performed additional experiments at 2 days of storage to assay the effectiveness of other potential sequestering compounds. We assayed activated carbon, ethanolamine, Zeolite 4A, lithium hydroxide (LiOH) and an activated magnesium silicate, FLORISTL®, at 100-200 mesh and less than 200 mesh. When measuring particle size. “mesh” is determined by the number of opening in one linear inch. For example, a screen of 200 mesh has two hundred openings in a linear inch whereas a 100 mesh screen has one hundred openings in a linear inch. Thus, powder at 200 mesh has finer particles than powder at 100 mesh. See, e.g., ASTM E11-20, Standard Specification for Woven Wire Test Sieve Cloth and Test Sieves, ASTM International, West Conshohocken, PA, 2020, www.astm.org. Soda lime and no-sequestrating agent treatments were used as controls. To assay the effectiveness of each CO₂ sequestering agent, between 0.02-9.6 g of agent were added to 125 mL glass jars containing 1 mL of water (Table 12). To mimic the typical amount of CO₂ released/g of pollen/day (Table 10), jars were sealed, vessel pressure was brought down to 0.9 atm, and then jars were backfilled with compressed CO₂ until internal pressure reached 1 atm. This resulted in about 0.4 mmol of CO₂ and about 0.95 mmol of oxygen per container. Containers were then placed at 6° C. for 2 days. After this period, containers were moved back to room temperature (approximately 21° C.) and final CO₂ and oxygen contents were measured (Table 12). Overall, all agents successfully sequestered some portion of the provided CO₂, though the amount required and efficiency of sequestration varied by compound.

TABLE 12
CO₂ sequestration ability of various chemical CO₂ sequestering
agents in containers starting at about 0.4 mmol of CO₂ and
about 0.95 mmol of oxygen per container.

| | Amount Agent | Remaining | Remaining |
|---|---|---|---|
| CO₂ Sequestering Agent | Used (g) | mmol O₂ | mmol CO₂ |
| Soda lime | 0.04 | 0.96 | 0.01 |
| Soda lime | 0.06 | 0.94 | 0.01 |
| Lithium hydroxide | 0.01 | 0.96 | 0.14 |
| Lithium hydroxide | 0.02 | 0.94 | 0.03 |
| Ethanolamine | 0.05 | 0.99 | 0.03 |
| Ethanolamine | 0.1 | 0.96 | 0.01 |
| Ethanolamine | 0.2 | 0.95 | 0.01 |
| FLORISIL ® <200 mesh | 3.6 | 0.91 | 0.04 |
| FLORISIL ® <200 mesh | 7.2 | 0.89 | 0.01 |
| FLORISIL ® 100-200 | 1.8 | 0.91 | 0.14 |
| mesh | | | |
| FLORISIL ® 100-200 | 3.6 | 0.91 | 0.07 |
| mesh | | | |
| FLORISIL ® 100-200 | 7.2 | 0.90 | 0.03 |
| mesh | | | |
| Zeolite 4A | 3.2 | 0.91 | 0.27 |
| Zeolite 4A | 6.4 | 0.87 | 0.19 |
| Zeolite 4A | 9.6 | 0.86 | 0.14 |
| Activated charcoal (100 | 3.2 | 0.90 | 0.14 |
| mesh) | | | |
| Activated charcoal (100 | 9.6 | 0.91 | 0.06 |
| mesh) | | | |
| Negative control | 0 | 0.98 | 0.40 |

Additionally, we tested the efficacy of mixing one of the sequestering compounds, FLORISIL®, directly with the pollen. In this experiment, 0.18 g of pollen was mixed directly with 0.09 g of FLORISIL® at <200 mesh. The pollen-FLORISIL® mixture was then placed in a 125 mL vessel, the vessel pressure brought down to 0.4 atm, and the vessel then placed at 6° C. for 5 days. After storage, the pollen-FLORISIL® mix was used in pollinations, with 0.5 mL of mix used for each pollination. The average seed set from these pollinations was 107 kernels with a standard deviation of 76 kernels. Thus, it is possible to mix the pollen directly with a CO₂ sequestering agent and still achieve seed set after storage.

A follow-up experiment was conducted to test the combined action of enhancing starting oxygen content and sequestering CO₂ produced during aerobic respiration. In this study, 0.9 g of pollen mixed with 0.45 g of talc was stored for 5 days in 125 mL vessels at 6° C. and 1 atm with 0.208 g soda lime and varying starting amounts of oxygen (Table 13). In contrast to the experiment from Table 7 above where 0.9 g of pollen exhausted the available 0.44 mmol of O₂ prior to the end of storage, the presence of excess oxygen in this experiment allowed the pollen to maintain the ability to germinate and set seed out to the tested 5 days. Neither metabolic rate nor PMC were affected by different oxygen treatments. Germination rate, however, appeared to decrease as starting oxygen concentrations increased. These results demonstrate the benefit of adjusting initial oxygen content and passive sequestration of respired CO₂ in our closed-loop system.

TABLE 13
Impact of different starting oxygen concentrations on stored pollen vigor traits.

| Start | | Start mmol | | Mean mmol O₂ | | |
|---|---|---|---|---|---|---|
| mmol | Start mmol | O₂/g | Mean | Used/g | | Germination |
| O₂ | O₂/g pollen | pollen/day | Seed Set | Pollen/Day | PMC | Rating |
| 1.08 | 1.20 | 0.24 | 391 | 0.18 | 47 | 5-4 |
| 1.45 | 1.61 | 0.32 | 417 | 0.20 | 47 | 4 |
| 1.81 | 2.01 | 0.40 | 347 | 0.19 | 46 | 3 |
| 2.22 | 2.47 | 0.49 | 289 | 0.22 | 48 | 2 |
| 2.58 | 2.87 | 0.57 | 145 | 0.20 | 46 | 2 |

Further testing included various experiments with different inbreds, hybrids, and storage duration conditions with a pressurized vessel storage system. Across these experiments, we evaluated absolute pressure (atm) up to 3 atm as well as the starting mmol of O₂ per liter of storage vessel headspace. As seen in Table 14 below, we were able to recover seed from stored pollen in conditions with sealed vessels pressurized up to 3 atm absolute pressure with standard atmospheric air or mixtures of standard atmospheric air and pure oxygen gas. Additionally, we recovered seed when storage pollen in vessels with a starting oxygen concentration of up to 27 mmol O₂ per liter vessel storage headspace.

TABLE 14
Average stored pollen performance using pollen stored in vessels
pressurized with standard atmospheric air or mixtures standard
atmospheric air and pure oxygen gas. The table shows comparisons
between the various conditions tested.

| | Absolute | | | | |
|---|---|---|---|---|---|
| | Pressure | Storage | Avg Seed | | |
| mmol O₂/L | (atm) | Duration | Set/Ear (k) | Stdev (k) | N= |
| 9 | 1 | 4 | 161 | 92 | 20 |
| 9 | 1 | 5 | 67 | 71 | 97 |
| 18 | 2 | 4 | 141 | 110 | 18 |
| 18 | 2 | 5 | 64 | 65 | 20 |
| 24 | 2 | 5 | 63 | 119 | 4 |
| 27 | 3 | 3 | 397 | 45 | 5 |

N = number of ears

### 5. Carrier Compounds

Crystalline quartz silica is a superior carrier for pollen storage than amorphous silicas and silicates. Existing examples of pollen storage technology use talc (hydrated magnesium silicate) or amorphous silicas (precipitated, pyrogenic, or silica gel) as a carrier to prevent pollen clumping. Synthetic amorphous activated magnesium silicate (e.g., FLORISIL®) may also be used as a carrier to prevent pollen clumping. While these existing carriers are effective at preventing pollen clumping by inhibiting interaction between the cell membranes of adjacent pollen grains, their structure may also inhibit interaction between pollen grains and silks during pollination using stored pollen. In addition, unique properties of amorphous silicas and silicates not found in crystalline silica are detrimental to pollen viability in storage.

Talc is a soft clay mineral with the chemical formula, Mg₃Si₄O₁₀(OH)₂, that occurs as foliated silicate sheets. These soft, foliated sheets break up upon mixing with pollen and completely coat the pollen surface. This coating inhibits both clumping interaction between pollen grains and the pollen grain to silk interactions required to initiate pollen tube germination. The act of mixing pollen with talc can decrease the potential seed set that can be generated by the pollen. Common alternatives to talc in pollen storage applications include amorphous forms of silica (precipitated, pyrogenic, or silica gel). Synthetic amorphous activated magnesium silicates (e.g., FLORISIL®) are also an effective carrier in pollen storage, depending on storage method used. All forms of silica share the chemical formula, SiO₂, while activated magnesium silicate is described by the chemical formula, MgO₃Si. Precipitated silica, silica gel, and synthetic amorphous magnesium silicates provide similar benefits to talc by inhibiting clumping interaction between pollen grains. However, these are detrimental to pollen viability in storage. These compounds have high specific surface areas and are shown to dehydrate pollen during storage. Pyrogenic silica is composed of low-density, polymer-like, silica agglomerates and is the most efficient at inhibiting clumping interaction between pollen grains. Pyrogenic silica also acts as a desiccant in storage and is detrimental to pollen viability. When pollen is mixed with pyrogenic silica, pollen grains are inhibited from binding to silks and germinating pollen tubes, thus pyrogenic silica eliminates the potential for fresh or stored pollen seed set to the same degree as if the pollen were non-viable. These desiccant, low mineral hardness, and high specific surface area properties make talc, amorphous silicas, and amorphous silicates ineffective carriers in pollen storage technology.

Silica is commonly found in nature as the crystalline mineral, quartz. Crystalline silica can exist in multiple polymorphic crystalline forms. Mixtures of these polyforms may be called polycrystalline silica. Crystalline silica has different structural properties than talc or synthetic amorphous silicas, which include but are not limited to a higher Mohs mineral hardness, higher bulk density, and lower specific surface area. Crystalline silica inhibits clumping interaction between pollen grains during storage but does not excessively coat pollen grains immediately upon application or due to carrier particle breakup during handling. In addition, crystalline silica does not act as a desiccant.

Pollen samples mixed with crystalline silica show more consistent, higher seed set following storage than talc (Tables 17, 18, and 19). A preferred average crystalline silica particle size is believed to be 10 μm (Table 19), but various applications may use particle sizes ranging between nanoparticles (e.g., 1 nm) to 100 μm. Pollen samples mixed with activated magnesium silicate show similar performance to pollen mixed with talc (Tables 16 and 20).

TABLE 15
Particle size, specific surface area, and
bulk density of pollen storage carriers.

| | Particle | | |
|---|---|---|---|
| | Size | Specific Surface | Bulk Density |
| Compound | (μm) | Area (m²/g) | (g/cm³) |
| Talc | <75 | 5-20 | 0.53 |
| Crystalline Silica | 0.5-10 | 0.04 | 0.78 |
| Crystalline Silica | 1 | 0.04 | 0.75 |
| Crystalline Silica | 10 | 0.04 | 1.17 |
| Crystalline Silica | 45 | 0.04 | 1.27 |
| Activated Magnesium Silicate | 75-149 | 280-300 | 0.54 |
| FLORISIL ® 100-200 mesh | | | |
| Activated Magnesium Silicate | <75 | 280-300 | 0.48 |
| FLORISIL ® <200 mesh | | | |
| Fumed Silica | 0.2-0.3 | 300-350 | 0.04 |

Table 15 describes key properties of pollen storage carriers that differentiate crystalline silica from other silicates. Values for particle size and specific surface area are sourced from manufacturer specifications. Bulk density was directly measured on carrier preparations used during pollen storage experiments.

TABLE 16
Fresh pollen seed set for three carriers.

| | | Days | Avg Seed | |
|---|---|---|---|---|
| Tester | Carrier | Stored | Set/Ear (k) | Stdev (k) |
| Inbred | Talc | 0 | 473 | 36 |
| | 10 μm Crystalline Silica | 0 | 419 | 26 |
| Hybrid | Talc | 0 | 594 | 49 |
| | Activated Magnesium Silicate | 0 | 570 | 74 |
| | FLORISIL ® <200 mesh | | | |

The industry standard has been to use talc as a carrier when working with fresh pollen. See, e.g., U.S. Pat. No. 2,570,511 (filed May 22, 1946). Crystalline silica and activated magnesium silicate both show similar performance to talc when working with fresh pollen. All carriers were mixed with pollen at a ratio of two parts pollen, one part carrier by weight. Pollinations were made within one hour onto receptive silks.

TABLE 17
Stored pollen seed set for two carriers.

| | | Avg Seed | |
|---|---|---|---|
| Carrier | Days Stored | Set/Ear (k) | Stdev (k) |
| Talc | 4 | 161 | 92 |
| 10 μm Crystalline Silica | 4 | 451 | 75 |

This experiment demonstrates that storing pollen with crystalline silica results in higher seed set than pollen stored with talc. Both carriers were mixed with pollen at a ratio of two parts pollen, one part carrier by weight. Pollinations were made after four days of storage onto receptive silks.

TABLE 18
Stored pollen seed set for two carriers.

| | | Avg Seed | |
|---|---|---|---|
| Carrier | Days Stored | Set/Ear (k) | Stdev (k) |
| Talc | 7 | 350 | 233 |
| 10 μm Crystalline Silica | 7 | 452 | 54 |

The standard deviations measured in this experiment further demonstrate that the use of a crystalline silicate carrier improves pollination performance by reducing the variation in seed set between ears. Both carriers were mixed with pollen at a ratio of two parts pollen, one part carrier by weight. Pollinations were made after seven days of storage onto receptive silks.

TABLE 19
Comparing various average sizes of crystalline SiO₂.

| | | Avg Seed | |
|---|---|---|---|
| Carrier | Days Stored | Set/Ear (k) | Stdev (k) |
| 1 μm Crystalline Silica | 7 | 224 | 132 |
| 10 μm Crystalline Silica | 7 | 302 | 4 |
| 45 μm Crystalline Silica | 7 | 34 | 14 |

This experiment demonstrates that 1 um or 10 um average particle size preparations of crystalline silica are the superior choice for storing corn pollen. The larger 45 um average particle size preparation successfully preserves pollen viability in storage and may be the superior choice for other pollen types. All carriers were mixed with pollen at a ratio of two parts pollen, one part carrier by weight. Pollinations were made after seven days of storage onto receptive silks.

Metallic powders are effective carriers for pollen storage that do not demonstrate the same disadvantages as talc powder and amorphous silicates. Metallic powders prevent clumping interaction between adjacent pollen grain membranes during storage but do not excessively coat the pollen membrane surface and do not inhibit adherence to maize silks or other plant stigmas. This lack of inhibition enables effective pollen tube germination and makes these carriers superior to talc powder and amorphous silicas. Elemental metallic powders, metallic oxide powders, and metallic carbide powders are all effective pollen storage carriers. These powders may be manufactured by diverse techniques to optimize function, including solid-state reduction, electrolysis, chemical reactions, high-temperature combustion, gas atomization, ultra-high pressure water atomization, pressing and sintering, centrifugal atomization, grinding, and other polishing techniques to optimize particle size and particle surface properties. The optimal particle type for metallic powders in maize pollen storage is believed to be 10 μm polished spherical particles, but other particle sizes and surface characteristics may be better for other pollen types. In some applications, metallic powders may be coated in polymers to modify particle surface interaction with pollen membranes. In other applications, metallic particles may be coated in active ingredients to modify interaction with pollen grain membranes, modify the respiration of pollen and microbes in storage, or inhibit microbial proliferation during storage. These active ingredients may include nucleic acids, proteins, pesticides, or bio-stimulants. Metallic carriers include elements with known biological roles in plants that may enhance pollen performance and those with no known biological role that have no impact on pollen performance. Ferro-magnetic carriers may be preferred in applications where the carrier can be magnetically removed from the pollen carrier mix following storage to enrich the concentration of pollen in the mix.

Micas are a group of minerals defined by a general chemical formula and perfect basal cleavage. Perfect basal cleavage results in flat sheet shaped particles that are effective in preventing interaction between adjacent pollen grain membranes. In addition to physical properties that make mica minerals effective carriers in pollen storage, the high reflectivity of mica minerals can act as a visual indicator during pollen application. These reflective properties can be visualized by protocol operators or machine cameras to track the distribution of pollen during application or verify where pollinations have taken place.

Table 20 details the performance of crystalline silica, metallic powder carriers, and mica as a carrier in pollen storage. All carriers in this test show similar performance to crystalline silica.

TABLE 20
Seed set from pollen stored with ten carriers.

| | | Pollen Tube | | |
|---|---|---|---|---|
| | Days | Germination | Avg Seed | Stdev |
| Carrier | Stored | Rating | Set/Ear (k) | (k) |
| 10 μm Crystalline Silica | 3 | 5 | 528 | 89 |
| 10 μm Mica | 3 | 5 | 598 | 33 |
| 10 μm Silicon Carbide | 3 | 5 | 519 | 59 |
| 10 μm 316L Stainless Steel | 3 | 5 | 466 | 27 |
| Powder | | | | |
| 10 μm 316L Stainless Steel | 3 | 5 | 423 | 150 |
| Powder Optimized for 3D | | | | |
| Printing | | | | |
| 10 μm Iron Powder | 3 | 5 | 487 | 39 |
| 10 μm Spherical Aluminum | 3 | 5 | 642 | 59 |
| 5 μm Spherical Aluminum | 3 | 5 | 540 | 126 |
| Oxide | | | | |
| 10 μm Titanium Powder | 3 | 5 | 515 | 28 |
| 10 μm Chromium Powder | 3 | 5 | 607 | 109 |

All carriers were mixed with pollen at a ratio of two parts pollen, one part carrier by weight. The 10 μm 316L stainless steel powder is produced through high-temperature combustion and individual particles have an amorphous structure. It is optimized for 3D printing through ultra-high-pressure water and gas atomization with grinding to produce uniform, spherical particles.

TABLE 21
Stored pollen seed set for two carriers
in two independent experiments.

| | | Days | Avg Seed | Stdev |
|---|---|---|---|---|
| Experiment | Carrier | Stored | Set/Ear (k) | (k) |
| 1 | Talc | 5 | 25 | 12 |
| | Activated Magnesium Silicate | 5 | 103 | 96 |
| | FLORISIL ® <200 mesh | | | |
| 2 | Talc | 5 | 107 | 107 |
| | Activated Magnesium Silicate | 5 | 4 | 4 |
| | FLORISIL ® <200 mesh | | | |

These experiments demonstrate the inconsistent outcomes that result from using talc or an activated magnesium silicate carrier in pollen stored for 5 days. Averages and standard deviations for experiment 2 are the same whole number after rounding. For both experiments, carriers were mixed with pollen at a ratio of two parts pollen, one part carrier by weight. Pollinations were made after five days of storage onto the same population of receptive silks.

### 6. Pollen Storage of Plants Containing Transgenic Events

Male ratings are an overall assessment of performance as a pollen source that accounts for all data types collected during inbred parent line development. Ratings from highest to lowest male performance are Desirable, Acceptable, Marginal, and Do Not Advance. Pollen for five inbred parent lines containing transgenic events that received overall male ratings of Acceptable, Marginal, or Do Not Advance was collected, separately, and mixed with a crystalline silica carrier at a ratio of two parts pollen, one part carrier by weight. A portion of the pollen mixed with crystalline silica was used to conduct fresh self-pollinations (stored zero days). The remaining pollen plus crystalline silica mix was stored in a sealed vessel with added soda lime in a 6° C. environment. After five days, stored pollen was applied to silks on the same inbred line that provided the pollen (i.e., a self-pollination). All ears received the same quantity of fresh pollen and carrier or stored pollen and carrier by volume.

TABLE 22
Seed set from stored pollen for five different inbred
parent lines. Inbred lines one through four comprise
transgenic events Bt11, GA21, and MIR162. Inbred line
five comprises transgenic events Bt11 and MIR162.

| | | Days | Avg Seed | |
|---|---|---|---|---|
| Pollen Source | Male Rating | Stored | Set/Ear (k) | Stdev (k) |
| Inbred Line 1 | Marginal | 0 | 301 | 47 |
| | | 5 | 36 | 33 |
| Inbred Line 2 | Acceptable | 0 | 53 | 22 |
| | | 5 | 92 | 48 |
| Inbred Line 3 | Marginal | 0 | 319 | 181 |
| | | 5 | 362 | 113 |
| Inbred Line 4 | Acceptable | 0 | 173 | 136 |
| | | 5 | 2 | 2 |
| Inbred Line 5 | Do Not Advance | 0 | 59 | 43 |
| | | 5 | 1 | 1 |

### Materials and Methods

### 1. MTT Staining

The use of 3-(4,5-dimethylthiazol-2-yl)-2,5-diphenyltetrazolium bromide (MTT) as a colorimetric assay for cell viability is common. Positive staining (visible purple coloring) of cells with MTT indicates activity of NAD(P)H-dependent cellular oxidoreductases and is thus an indication of metabolic activity of a living cell. In our studies, 200 uL of a 0.9% MTT, 5% sucrose solution is added to ˜20 uL of pollen, left to sit covered at room temperature for 5 min, and then stain color and intensity are recorded. Enzymatic activity is one of the last processes to halt in dying pollen and is a reliable indicator of whether the tissue is truly dead (in which case one observes a complete lack of staining). Positive staining should be combined with other assays, such as pollen tube germination and pollinations, to fully assay the vigor of the pollen and its ability to fertilize.

### 2. Pollen Tube Germination

Prior to fertilization of the embryo, maize pollen must germinate a tube through the silks to deliver the sperm cells to the ovary. This ability to germinate pollen tubes can be assayed on solid media. Pollen is sprinkled on the surface of the media, allowed to sit on the bench at room temperature for 60 min, and then observed or imaged. The number of pollen grains with tubes is then counted or scored. While quantitative scoring of germinated pollen grains is possible, it is highly time consuming. As an alternative, a ranking on a categorical scale can be assigned to denote the extent of tube germination/germinability for a given pollen sample. In our studies, we used a scale from 1-5 where 1=no germination, 2=1-20% of grains germinating, 3=21-40% of grains germinating, 4=41-60% of grains germinating, 5=>60% of grains germinating. We counted any pollen grain with a tube greater than or equal in length to the diameter of a single grain after 60 min of incubation as successfully germinated.

3. Pollinating with Stored Pollen and Measuring Seed Set.

There are several tools that can be used to understand the approximate viability of stored pollen. These tools include MTT staining, pollen tube germination, and impedance flow cytometry devices such as Ampha Z32 (made by Amphasys; amphasys.com/ampha-z32-pollen-analyzer/). Each of these tools has their uses, and when used in combination may provide a reasonable estimation of the ability of pollen to fertilize ovules. However, measuring seed set remains the strongest indicator of this trait.

Seed set measured in the experiments documented here was generated from controlled greenhouse or field pollinations. Ears used for pollinations were bagged prior to silking to prevent contamination from airborne pollen. Pollen used in storage was typically collected from multiple tassels of the same line and then bulked into a single batch of pollen. Each batch was thoroughly mixed before being distributed into individual sample vessels. After storage, pollen can either be directly applied to ears from the storage container or bulked again and subsamples used for pollination. Pollination is done by hand—ear bags are removed long enough to conduct the hand pollination and then ears are covered with a larger ear bag to prevent contamination from outside pollen sources.

Kernels are then allowed to develop for 12-14 days when ears are harvested for kernel counting. Kernels are counted by hand or by image analysis software. Aborted kernels are not included in the kernel count.

What is claimed is:

1. A composition comprising maize pollen and crystalline silica.

2. (canceled)

3. The composition of claim 1, wherein the average particle size is between about 1 nanometer and about 100 micrometers, or between about 1 micrometer and about 10 micrometers.

4. (canceled)

5. The composition of claim 1, wherein the maize pollen is 0 days old, 1 day old, 2 days old, 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, or more.

6. A method of storing viable maize pollen, comprising:
a) collecting an amount of fresh maize pollen;
b) applying a carrier to the collected maize pollen of step a) to obtain an amount of treated maize pollen;
c) placing the amount of fresh maize pollen or the amount of treated maize pollen in a sealable container and optionally setting a vessel pressure; and
d) storing the product of step c) in a refrigerated environment, wherein the stored maize pollen remains viable for at least 1, 2, 3, 4, 5, 6, 7, 8, 9, 10, 11, 12, 13, 14, 15, 16, 17, 18, 19, or 20 days.

7. (canceled)

8. The method of claim 6, wherein the container comprises a volume of 1 mL to 100 L.

9. (canceled)

10. (canceled)

11. The method of claim 6, wherein the amount of fresh maize pollen or treated maize pollen is approximately 1-54 g.

12. (canceled)

13. (canceled)

14. (canceled)

15. (canceled)

16. (canceled)

17. (canceled)

18. (canceled)

19. (canceled)

20. The method of claim 6, wherein the vessel pressure is between approximately 0.6 atm and 0.3 atm.

21. (canceled)

22. The method of claim 6, wherein the carrier is selected from the group consisting of crystalline silica, activated magnesium silicate, talc, metallic powder, and mica mineral.

23. The method of claim 22, wherein the metallic powder is a metallic oxide powder or a metallic carbide powder.

24. (canceled)

25. The metallic powder of claim 24, wherein the metallic powder particle size is 10 μm spherical on average.

26. The metallic powder of claim 23, wherein the metallic powder is stainless steel powder.

27. The method of claim 6, wherein the carrier is present in a pollen:carrier ratio selected from the group consisting of 1:20, 1:30, 1:10, 1:5, 1:3, 1:2, 1:1, 2:1, 3:1, 4:1, 5:1, 6:1, 7:1, 8:1, 9:1, 10:1, 20:1, 30:1, 40:1, 50:1, and any ratio between 1:20 and 50:1.

28. (canceled)

29. The method of claim 6, wherein the sealable container comprises an aluminum tray, copper tray, nickel tray, or a stainless-steel tray platform.

30. (canceled)

31. (canceled)

32. The method of claim 6, wherein the sealable container is fabricated from glass, acrylic, aluminum, or stainless-steel.

33. (canceled)

34. The method of claim 6, wherein the refrigerated environment comprises a temperature range selected from the group consisting of 1° C.-10° C., 4° C.-8° C., and 5.5° C.-6.5° C.

35. (canceled)

36. The method of claim 6, wherein the pollen is stored in the refrigerated environment for 20 or fewer days, 19 or fewer days, 18 or fewer days, 17 or fewer days, 16 or fewer days, 15 or fewer days, 14 or fewer days, 13 or fewer days, 12 or fewer days, 11 or fewer days, 10 or fewer days, 9 or fewer days, 8 or fewer days, 7 or fewer days, 6 or fewer days, 5 or fewer days, 4 or fewer days, 3 or fewer days, 2 or fewer days, or 1 day, or less than 1 day.

37. (canceled)

38. The method of claim 6, wherein the sealable container comprises a starting oxygen content between 0.12 mmol O₂/q pollen/day stored and 0.57 mmol O₂/q pollen/day stored.

39. (canceled)

40. (canceled)

41. The method of claim 6, wherein the sealable container comprises a CO₂ sequestration agent selected from the group consisting of activated charcoal, ethanolamine, Zeolite 4A, lithium hydroxide (LiOH), soda lime, calcium silicate (Ca₂O₄Si), and activated magnesium silicate (e.g., FLORISIL®).

42. (canceled)

43. The method of claim 22, wherein the crystalline silica comprises an average particle size between about 1 nanometer and about 100 micrometers, or between about 1 micrometer and about 10 micrometers.

44. (canceled)

45. (canceled)

46. A method of applying stored maize pollen to a stigma, comprising:
a) obtaining stored maize pollen by the method of claim 6;
b) applying the stored pollen to a stigma;
wherein the stored maize pollen is applied to the stigma after collection.

47. The maize pollen of claim 46, wherein the maize pollen is applied to the stigma at least 1 day after collection.

48. The method of claim 46, wherein the stigma is a maize silk.

49. The method of claim 48, wherein the maize silk is a different heterotic group than the heterotic group corresponding to the stored maize pollen.

50. The method of claim 49, wherein the maize silk is from a tropical or sub-tropical heterotic group and the stored maize pollen is from a temperate heterotic group; or the maize silk is from a temperate heterotic group and the stored maize pollen is from a tropical or sub-tropical heterotic group, wherein the heterotic group is selected from the group consisting of Stiff Stalk, Non-Stiff Stalk, Iodent, and Lancaster.

51. (canceled)

52. The method of claim 48, wherein the maize silk is a different maturity group than the maturity group corresponding to the stored maize pollen.

53. The method of claim 47, wherein the stigma is a wheat stigma.

54. A method of accelerated trait introgression in the genome of a plant, the method comprising:
a) providing a first plant being of a first maturity group;
b) cross pollinating the first plant of (a) with stored pollen from a second plant being of a second maturity group and further having a desired trait or phenotype; and,
c) selecting a progeny plant from step (b) comprising the desired trait or phenotype; and
d) optionally, backcrossing the progeny plant of (c) as the pollen donor onto a recurrent parent plant and selecting progeny plants comprising the desired trait or phenotype,
wherein the stored pollen is obtained by the method of claim 6.

55. The method of claim 54, wherein the plant is a maize plant.

56. The method of claim 54, wherein the first maturity group is greater than one maturity group away from the second maturity group.

57. The method of claim 54, wherein the stored pollen from the second plant is applied to the first plant at least 1 day after collection.

58. The method of claim 6, wherein the maize pollen is transgenic maize pollen comprising at least 1 transgenic event selected from the group consisting of MIR162, Bt11, GA21, MIR604, MZIR098, 5307, 3272, DAS40278, TC1507, DAS-59122-7, NK603, MON810, MON863, MON89034, MON88017, DP-4114, and MON87411.

59. (canceled)

60. (canceled)

61. (canceled)

62. (canceled)

63. The method of claim 6, wherein the vessel pressure is pressurized with standard atmospheric air or a mixture of standard atmospheric air and pure oxygen gas, wherein the standard atmospheric air is 1-3 atm absolute and the pure oxygen gas in 18-27 mmol O₂ per liter of storage vessel headspace.

64. (canceled)

65. (canceled)

66. (canceled)

67. (canceled)

68. (canceled)

69. (canceled)
